(19) United States
(12) Patent Application Publication
(10) Pub. No.: US 2009/0306500 A1
(43) Pub. Date: Dec. 10, 2009

(54) WORKFLOW FOR MINIMALLY INVASIVE HEART TREATMENT

(76) Inventors: Norbert Rahn, Forchheim (DE); Erik Busch, Hemhofen (DE); Alois Noettling, Pottenstein (DE)

Correspondence Address: BRINKS HOFER GILSON & LIONE, P.O. BOX 10395, CHICAGO, IL, 60610, US

(21) Appl. No.: 12/239,300

(22) Filed: Sep. 26, 2008

Related U.S. Application Data
(60) Provisional application No. 61/059,352, filed on Jun. 6, 2008.

(51) Int. Cl. A61B 6/00; A61F 2/24; A61B 5/0402; G06K 9/00

(52) U.S. Cl. 600/431; 600/436; 623/2.11; 600/509; 382/131

(57) ABSTRACT

A system and method of treating a patient is described, where an implantable device is introduced into the patient and guided to an appropriate location using a 2-dimentsional X ray taken prior to the introduction of the device, and a fluoroscopic image taken from the same aspect during the procedure, and using the same portion of a physiological cycle. The implantable device may be a percutaneous aortic heart valve (PHV), and the location of the device may be determined with respect to specific bodily structures identified in the 2-dimensional X-ray, such as the aortic valve and the coronary ostia. The installation position of the device is selected so as to avoid obstruction of the coronary ostia.

This application claims the benefit of U.S. provisional application Ser. No. 61/059,352, filed on Jun. 6, 2008, which is incorporated herein by reference.

### TECHNICAL FIELD

The present application generally relates to the use of medical imaging to guide the placement of an implantable device. More particularly the application relates to a minimally invasive procedure for replacement of a defective human heart valve.

### BACKGROUND

Heart valve replacement (in particular replacement the aortic valve) is most often performed as an invasive surgical procedure by opening the rib cage. Aortic valve replacement is most frequently done through a median sternotomy; that is, the breastbone is sawed in half to provide access to the heart. Once the pericardium has been opened, the patient is placed on a cardiopulmonary bypass machine, also referred to as the heart-lung machine. An incision is made in the aorta; the surgeon then removes the diseased heart valve and a mechanical, processed biological, or autograft tissue valve is inserted and secured. A transesophageal echocardiogram (TEE, an ultra-sound of the heart performed through the esophagus) can be used to verify that the new valve is functioning properly.

Other treatment procedures are being developed, including minimally invasive techniques using catheter systems. For example, in a transapical valve replacement, an artificial heart valve is introduced through a tube, which is inserted in a minimally invasive fashion through the rib cage and through the myocardium at the apex of the heart, and the valve maneuvered into place through the use of X-ray control. In a transfemoral approach, an artificial heart valve is introduced via the aorta using a catheter and is maneuvered into place through the use of X-ray control.

The selection of a particular medical treatment is a matter of professional judgment, based on the specific nature of the medical syndrome, patient condition and factors including the maturity and risks associated with potential courses of treatment.

In a minimally invasive percutaneous method, a sheath, or introducer, is inserted into a blood vessel exposed by an incision. The sheath is a plastic tube through which a catheter will be inserted into the blood vessel and advanced into the heart. This may be done by obtaining fluoroscopic (real-time X-ray) images, and using the images so as to assist in guiding the catheter to the proper position. The catheter may be advanced into either the right or left side of the heart, or both sides, depending on particular valve to be replaced.

An example of an “artificial” heart valve is one made from three leaflets of animal pericardium (for example, bovine or equine) sutured to a balloon-expandable stainless-steel stent. In the procedure, a balloon catheter may be used to dilate the existing valve. The artificial heart valve may be crimped over a balloon catheter, and advanced over a stiff guidewire through the blood vessels (for example, from the femoral vein: the antegrade/transseptal approach; or, the femoral artery: the retrograde approach) up to the diseased valve and positioned with respect to the existing diseased valve. The artificial valve may then be secured in place by the balloon expansion of the artificial valve, which may also include a stent so as to maintain the dilation of the insertion region.

Such a percutaneous aortic heart valve (PHV) is a trileaflet bovine pericardial valve, which is mounted within a stainless steel tubular slotted stent having a height of 14.5 mm and an external diameter, when expanded, of either 23 or 26 mm (Edwards Lifesciences, Irvine, Calif.). Other similar PHV products are being developed by CoreValve, Irvine, Calif.

When the aortic valve is being replaced, the arteries that branch from the aorta immediately above the aortic valve, require special consideration during the procedure. In particular it is necessary to ensure that the heart valve replacement does not lead to a closure or obstruction of the coronary ostia, which are the two openings in the aortic sinus that mark the origin of the (left and right) coronary arteries.

### SUMMARY

A method treatment of a patient by minimally invasive intervention is described, the method including: providing an imaging modality and equipment for performing minimally invasive therapy; positioning the patient in the treatment room so that radiographic image data may be obtained using the imaging modality; and, processing the radiographic image data so as to select a suitable orientation of the imaging modality with respect to the patient. A radiographic image taken in the suitable orientation is used as a first image. An implantable device is inserted into the patient and guided using a merged display of the first image with a fluoroscopic image of the patient obtained during the guiding procedure. The relationship of an aspect of the fluoroscopic image identified as the implantable device may be used to position the implantable device with respect to patient bodily structures identified in the first image.

In an aspect, a system for treating a patient is described, the system including a C-arm X-ray device, a catheter system, and an electrocardiograph. A catheter of the catheter system is capable of introducing and implanting a device in the patient. The C-arm X-ray device is operated to produce a first 2-dimensional image, and the C-arm X-ray device is operated to produce a fluoroscopic image obtained from the same aspect as the first image and at substantially a same state of a cardiac cycle of a patient, so that a location of the implantable device with respect to an identified internal bodily structure of the patient may be determined.

### BRIEF DESCRIPTION OF THE DRAWINGS

FIG. 1 is a block diagram illustrating a treatment system; and

FIGS. 2A and 2B show a block diagram illustrating a method of implanting a device in a patient body.

### DESCRIPTION

Exemplary embodiments may be better understood with reference to the drawings, but these embodiments are not intended to be of a limiting nature. Like numbered elements in the same or different drawings perform similar functions.

The combination of hardware and software to accomplish the tasks described herein may be termed a platform, treatment suite, system, or the like. The instructions for implementing processes of the platform may be provided on computer-readable storage media or memories, such as a cache, buffer, RAM, removable media, hard drive or other computer readable storage media. Computer readable storage media include various types of volatile and nonvolatile storage media. The functions, acts or tasks illustrated or described herein may be executed in response to one or more sets of instructions stored in or on computer readable storage media. The functions, acts or tasks may be independent of the particular type of instruction set, storage media, processor or processing strategy and may be performed by software, hardware, integrated circuits, firmware, micro code and the like, operating alone or in combination. Some aspects of the functions, acts, or tasks may be performed by dedicated hardware, or manually by an operator.

In an embodiment, the instructions may be stored on a removable media device for reading by local or remote systems. In other embodiments, the instructions may be stored in a remote location for transfer through a computer network, a local or wide area network, by wireless techniques, or over telephone lines. In yet other embodiments, the instructions are stored within a particular computer, system, or device.

Where the term “data network”, “web” or “Internet”, or the like, is used, the intent is to describe an internetworking environment, which may include both local and wide area telecommunications networks, where defined transmission protocols are used to facilitate communications between diverse, possibly geographically dispersed, entities. An example of such an environment is the world-wide-web (WWW) and the use of the TCP/IP data packet protocol, and the use of Ethernet or other known or later developed hardware and software protocols for some of the data paths. Often, the internetworking environment is provided, in whole or in part, as an attribute of the facility in which the platform is located and may be provided by others, or shared with other users.

Communications between the devices, systems and applications may be by the use of either wired or wireless connections. Wireless communication may include, audio, radio, lightwave or other technique not requiring a physical connection between a transmitting device and a corresponding receiving device. While the communication may be described as being from a transmitter to a receiver, this does not exclude the reverse path, and a wireless communications device may include both transmitting and receiving functions. Such wireless communication may be performed by electronic devices capable of modulating data as a signal on a carrier wave for transmission, and receiving and demodulating such signals to recover the data. The devices may be compatible with an industry standard protocol such as IEEE 802.11b/g, or other protocols that exist, or may be developed.

The terms used herein are believed to be, and are meant to be interpreted as, understood by a person of skill in the art at the time of preparation of the specification, unless specifically differentiated herein.

When describing a medical intervention technique, the terms “non-invasive,” “minimally invasive,” and “invasive” may be used. Generally, the term non-invasive means the administering of a treatment or medication while not introducing any treatment apparatus into the vascular system or opening a bodily cavity. Included in this definition is the administering of substances such as contrast agents using a needle or port into the vascular system. Minimally invasive means the administering of treatment or medication by introducing a device or apparatus through a small aperture in the skin into the vascular or related bodily structures. This includes the treatments known as percutaneous transluminal coronary angioplasty (PCTA), balloon angioplasty, stenting, and the like. Other minimally invasive techniques may be provide direct access to an organ through a small incision. Invasive techniques may include conventional surgery such as coronary artery bypass graft surgery (CABG), and the like.

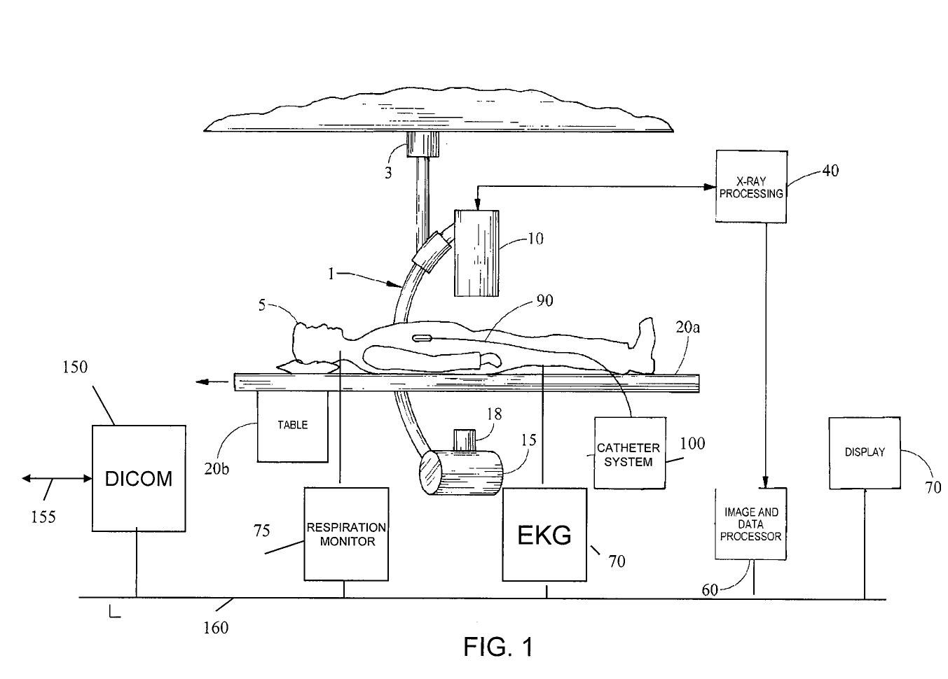

FIG. 1 illustrates a treatment suite which may be used to perform minimally invasive replacement of a heart valve by implantation of a device. The equipment may include a patient support table 20a, which may be positioned so as to be accessible to an X-ray device, which may be a C-arm X-ray device 1, so that 2D digital radiographs may be obtained at selectable orientations of the X-ray device 1 with respect to the patient 5. Other equipment may include a catheter system 100 for performing the minimally invasive procedure, for performing angiograms, or other uses; an electrocardiograph (EKG) 70 in communication with a computer 60 for controlling the X-ray device 1, and one or more image displays 70. Other life support and monitoring equipment, as is known in the art, may be present and be used.

Where the term “catheter” is used, it is intended to represent any treatment apparatus introduced into the patient's body, and may also include, for example, the capability of dispensing contrast agents introduced intra-operatively to visualize the results of a procedure, or positioning a device for implantation.

The C-arm device X-ray 1 imaging modality may comprise an X-ray tube 15, high-voltage power supply, radiation aperture 18, X-ray detector 10, digital imaging system 40, and system controller, as well as user control and display units 70. The X-ray detector 10 may be amorphous Selenium (a-Se), PbI2, CdTe or HgI2 detectors using direct detection and TFT technology, or indirect detectors as is known in the art, or may be subsequently be developed, to provide high resolution, high-dynamic-range real-time X-ray detection. The X-ray detector may be disposed diametrically opposed to the X-ray source and such that the plane of the detector is perpendicular to the axis of the X-ray source. This orientation may, for example, be maintained by attaching the X-ray source and X-ray detector to a C-arm, a U-arm or the like. The C-arm may be mounted to a robot 3 so as to permit the X-ray source and detector to be oriented with respect to the patient.

The C-arm X-ray device may be operated to obtain fluoroscopic images, or data suitable for the production of 2D images.

A patient 5 may be positioned on a patient support apparatus 20a. The patient support apparatus 20a may be a stretcher, gurney, or the like, and may be attached to a robot 20b. The patient support apparatus 20a may also be attached to a fixed support or adapted to be removably attached to the robot. Aspects of the patient support apparatus 20a may be manipulable by the robot 20b. Additional, different, or fewer components may be provided.

The data processing and system control is shown as an example, and many other physical and logical arrangements of components such as computers, signal processors, memories, displays and user interfaces are equally possible to perform the same or similar functions. The particular arrangement shown is convenient for explaining the functionality of the system.

The devices and functions shown are representative, but not inclusive. The individual units, devices, or functions may communicate with each other over cables, over a local or wide area network, or in a wireless manner. The various devices may communicate with a DICOM (Digital Communications in Medicine) system 150 and with external devices over a network interface 155, so as to store and retrieve image and other patient data. Local communication may over a LAN 160. Images reconstructed from the X-ray data may be stored in a non-volatile (persistent) storage device, which may be a part of the processor 60, or be stored in auxiliary storage or transmitted over a network, for further use. The X-ray device 1 and the image processing attendant thereto may be controlled by a separate controller 60 or the function may be consolidated with the user interface and display 70.

The X-ray images may be obtained with or without various contrast agents that are appropriate to the imaging technology and diagnosis protocol being used.

The physiological sensors, which may be an electrocardiograph (EKG) 70, a respiration sensor 75, or the like, may be used to monitor the patient 5 so as to enable selection of radiographic and fluoroscopic images that represent a particular portion of a cardiac or respiratory cycle as a means of minimizing motion artifacts in the images.

The positioning of a catheter inside a patient, and the manipulation of the catheter position to administer treatment or perform a procedure is facilitated by the use of real-time fluoroscopic images of the patient. Alternatively, the position of the catheter or other apparatus may be measured by acoustic or magnetic means, and superimposed on a fluoroscopic or 2-D X-ray image, and the image may be a previously captured image, or based on previously acquired data.

When fluoroscopy alone is used, a small wire may not be easily visualized in the distracting background image of the underlying tissue. Image enhancement techniques may be used to assist the operator. Image subtraction and roadmap imagery are known. The image may be produced by first obtaining a 2-D image data set of the patient in the same position as treatment will be administered, by administering a contrast agent so as to visualize the structure to be treated, and by making a composite image of a series of images taken during the administration of the contrast agent, so as to produce a mask image.

The X-ray image is formed by detection of X-rays that have been attenuated exponentially in passing through the body. Subtraction of pre- and post-contrast images take this exponential attenuation into account by using logarithmic subtraction. The process is known as Digital Subtraction Angiography (DSA).

The brightness of the objects in the subtracted angiographic image (e.g., the vessels with contrast material) is not substantially affected by the brightness (density) of the underlying tissues in the non-subtracted images. The X-ray beam is not mono-energetic, the logarithmic subtraction is not perfect; and there may still be a slight variation of vessel brightness that is dependent on the attenuation of the underlying tissue.

When performing the placement step of a procedure to replace a heart valve using a minimally invasive procedure, the position the replacement heart valve in relation to the aortic root must be closely monitored during the guidance/placement thereof in order to establish an optimal position of the artificial heart valve before it is fixed in place. In particular, the replacement heart valve and any associated structure, such as a stent, must not lead to a closure of the coronary ostia. The coronary ostium are either of the two openings in the aortic sinus that mark the origin of the (left and right) coronary arteries. As the coronary arteries are the source of supply of oxygenated blood to the heart itself, the function of the coronary arteries should not be impaired by placement of the replacement heart valve structure.

Consequently, during the guidance and placement of the replacement heart valve, the replacement heart valve must also be substantially continuously visualized in relation to the coronary arteries arising from the aorta. The procedure may be performed while the heart is beating and, in order to achieve a suitable matching of the imaging of the artificial heart valve during its guidance/placement, heartbeat and respiration-induced influences on the imaging should be minimized. Synchronization of the fluoroscopic images with previously obtained roadmap or DSA images obtained with the same orientation as now being used during the procedure may be facilitated by the use of an EKG (electrocardiogram) and a breath monitor. Generally, the patient will be requested to hold the breath (inhale or exhale stage) and a specific stage of the cardiac cycle, measured by, or monitored by, the EKG may be used to obtain the real-time fluoroscopic image so that it corresponds to the same or similar physiological conditions as the roadmap, angiographic or other previously obtained 2-D X-ray images.

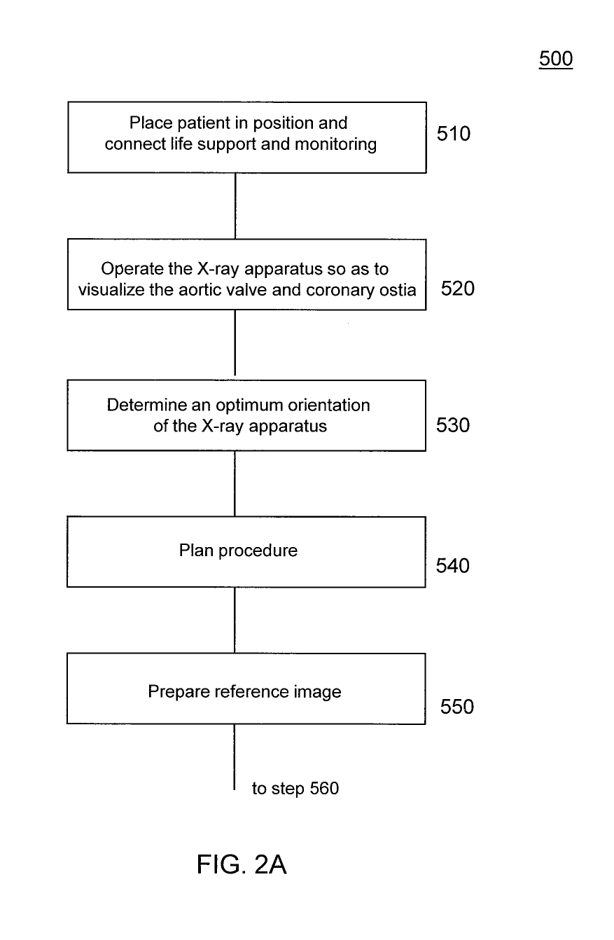

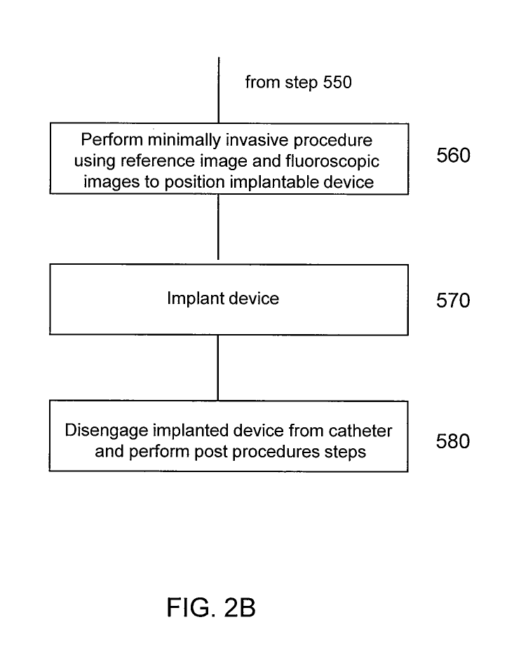

An example of the method and workflow 500, using the apparatus of FIG. 1 to perform transfemoral valve replacement, and the corresponding workflows, are summarized in FIG. 2 A-B. Preparatory to the guidance and placement of the replacement heart valve, a step (510) of placing the patient on the support structure associated with the X-ray apparatus and connecting any monitoring and life support devices is performed. The patient would be placed in a position in which the minimally invasive procedure may subsequently be performed. The C-arm X-ray device is operated so as to produce 2-D images of the patient so that the aortic valve and the coronary ostia may be visualized (for the replacement of the aortic valve) (step 520). A contrast agent may be injected into the left ventricle in the vicinity of the aortic root and a plurality of X-ray images are obtained and recorded so as to determine an optimum position of the C-arm (step 530) for this visualization. The X-ray images may be associated with EKG and respiration data so that the images associated with a specific phase of the patient physiological cycle may be obtained, selected or displayed. In an aspect, the images may be obtained at a preselected phase of the physiological cycle. Based on these images, quantitative measurements may be obtained, such as thickness of the aortic lumen for device selection, and distance of the coronary ostia from the aortic root for the planning of the valve replacement (step 540). A 2-D reference image may be formed using the images obtained in the optimum position and using the injected contrast agent so as to produce DR or DSA images. (step 550). In an aspect, the images may be displayed in an inverted gray scale. The contrasted vessels may depicted as dark (high attenuation) and the background of the image is depicted as light. Subsequently when an image of the catheter and the replacement heart valve is digitally superimposed (step 560), the image of the catheter and replacement valve would be light and be visible against the previously obtained radiographic image with contrast. Of course, non-contrast enhanced images may also be used. The gray scale threshold and gamma may be adjusted to achieve suitable contrast. The sense of the catheter and angiographic images may be inverted.

The reference image is taken at a definite cardiac phase (for example 70% of the R-R interval as indicated on the electrocardiogram), and while the patient holds his breath in a definite breath-holding phase (e.g. expiration).

The patient is further prepared, as needed, for the minimally invasive treatment (step 560). The catheter is introduced into the patient, and 2-D fluoroscopic images are obtained during the guidance and placement of the artificial heart valve; the images are overlaid on the reference images obtained previously (step 550). In an aspect, the fluoroscopic images may be EKG triggered at the same heart cycle phase that was used to generate the 2-D reference image so as to reduce the number of X-ray images actually needed, and reduce the cumulative X-ray does to the patient. The percentage of image overlay can be predetermined or may be changed by means of a user interface (e.g. a joystick) mounted so as to be accessible to the physician. During the procedure, it may be additionally needed for the patient to be brought into the same breath-holding phase as that which obtained during the production of the reference images. This is usually done by a verbal request to the patient, when the patient is conscious. A respiration monitor may be used if, for example, the patient is unresponsive.

As the heart-valve-replacement device is being positioned, the position of the device is monitored with respect to the existing natural aortic valve, and with respect to the coronary ostia. The device may be positioned so that, when the balloon is used to expand the stent and emplace the valve, the emplaced valve will be properly located so as to avoid obstructing the ostia and to be in a correct position with respect to the ventricle. The stent may be expanded so as to emplace the replacement valve (step 570). Once the replacement valve has been emplaced, the catheter may be disengaged from the emplaced valve, and extracted from the patient (step 580). The remainder of the procedure for securing the incision and other post-operative steps are known.

In an aspect, the use of computer image processing may be used so as to identify the coronary ostia and to provide additional graphical markings on the images. Additionally, the spatial orientation of the X-ray device with respect to the patient may be used to mark, for example, the cross sectional plane of the aortic valve. Further, the replacement valve image may be analyzed so as to clearly identify and enhance the image of the replacement valve, so as to make the superimposition of the images more effective to view. Alternatively, the ostia may be marked on the reference image manually. A marker may be placed on the image so that when a reference designation on the replacement valve is approaching a correct position or is positioned correctly, a signal, which may be an acoustical or optical signal, is given. Alternatively, such signals may be used to warn the physician that the device is in a region that is inappropriate for implantation.

The method has been described where the determined optimum C-arm position is used throughout the procedure. However, it may be necessary to change the orientation. Should such a change be needed, the reference 2-D X-ray may need to be generated again using steps 520 and 530

In patients with significant heartbeat variability, imprecisions in the image overlay (fluoroscopic images with reference image) may occur since the EKG triggering of the imaging of the reference image and the radioscopic image may not assure precisely identical states with regard to cardiac motion. Particularly in these cases, the cardiac motion may be stopped both during generation of the reference image and in significant phases of the procedure (e.g., placement of the heart valve) by means of rapid ventricular pacing. Alternatively, the cardiac motion may be stopped by administering adenosine during generation of the reference image and in the significant phases of the procedure.

While the methods disclosed herein have been described and shown with reference to particular steps performed in a particular order, it will be understood that these steps may be combined, sub-divided, or reordered to from an equivalent method without departing from the teachings of the present invention. Accordingly, unless specifically claimed herein, the order and grouping of steps and the parametric values are not a limitation of the present invention.

The examples of diseases, syndromes, conditions, and the like, and the types of examination and treatment protocols described herein are by way of example, and are not meant to suggest that the method and apparatus is limited to those named, or the equivalents thereof. As the medical arts are continually advancing, the use of the methods and apparatus described herein may be expected to encompass a broader scope in the diagnosis and treatment of patients.

Apart from the sensors positioning and catheterization capabilities, the imaging, data processing, and controlling equipment may be located within the treatment room or remotely, and the remotely-located equipment may be connected to the treatment room by a telecommunications network. Aspects of the diagnosis and treatment may be performed without personnel, except for the patient, being present in any of the local treatment rooms.

It is intended that the foregoing description be regarded as illustrative rather than limiting, and that it be understood that it is the following claims, including all equivalents, that are intended to define the spirit and scope of this invention.

What is claimed is:

1. A method treatment of a patient by minimally invasive intervention, the method comprising:
providing an imaging modality and equipment for performing minimally invasive treatment;
positioning the patient so that radiographic image data are obtained using the imaging modality;
processing the radiographic image data so as to select a suitable orientation of the imaging modality with respect to the patient;
using a radiographic image taken in the suitable orientation as a first image;
inserting an implantable device into the patient;
guiding the implantable device using a merging of the first image with a fluoroscopic image of the patient obtained during the guiding procedure; and
using the relationship of an aspect of the fluoroscopic image identified as the implantable device to position the implantable device with respect to patient bodily structures identified in the first image.

2. The method of claim 1, further comprising administering a contrast agent when obtaining the first image.

3. The method of claim 1, wherein the identifiable bodily structures are the coronary ostia of the aorta, and the implantable device is disposed so that it may be implanted without obstructing the coronary ostia.

4. The method of claim 3, wherein the implantable device is a percutaneous aortic heart valve.

5. The method of claim 1, wherein the treatment equipment includes a electrocardiograph (EKG) and the EKG is used to select a same phase of a heart cycle for the fluoroscopic image as was used for the first image.

6. The method of claim 1, wherein a new first image is obtained when the orientation of the imaging modality with respect to the patient is changed, and the new first image replaces the first image.

7. The method of claim 1, wherein the imaging modality is a C-arm X-ray device.

8. The method of claim 7, wherein a gray scale of the first image is inverted with respect to a gray scale of the fluoroscopic image, and the first image and the fluoroscopic image are superimposed for display.

9. The method of claim 1, wherein the patient bodily structures identified are the aortic valve and the coronary ostia.

10. The method of claim 1, further comprising:
guiding a catheter having an inflatable balloon to a position so as to be capable of engaging the aortic valve, and inflating the balloon; the step being performed prior to a step of implanting the implantable device.

11. The method of claim 1, wherein the implantable device is introduced into the patient using a catheter.

12. The method of claim 1, wherein the first image and the fluoroscopic image are obtained at a same respiratory state of the patient.

13. The method of claim 11, wherein the first image and the fluoroscopic image are obtained at a substantially same place in a cardiac cycle of the patient.

14. A system for treating a patient, comprising:
a C-arm X-ray device;
a catheter system, a first catheter thereof capable of introducing and implanting a device in the patient; and
an electrocardiograph (EKG);
wherein the C-arm X-ray device is operated to produce a first image, which is a 2-dimensional image, and the C-arm X-ray device is operated to produce a second image, which is a fluoroscopic image obtained from a same aspect as the first image and at a substantially same state of a cardiac cycle of a patient, so that a location of the implantable device with respect to an identified internal bodily structure of the patient may be determined.

15. The system of claim 14, wherein a second catheter has an expandable balloon.

16. The system of claim 14, wherein a third catheter is configured to administer a radio-opaque contrast agent.

17. The system of claim 14, wherein the identified bodily structure comprises an aortic valve and two coronary ostia.

18. The system of claim 14, wherein an audible or visual indication is provided when the implantable device is within a predetermined distance with respect to the identified bodily structure.

19. The system of claim 14, wherein a gray scale of the first image is inverted with respect to a gray scale of the second image, and the images are superimposed for display.

20. The system of claim 14, wherein an audible or visual indication is provided when the implantable device is closer than a predetermined distance with respect to the identified bodily structure.
